(19) United States
(12) Patent Application Publication
(10) Pub. No.: US 2006/0134015 A1
(43) Pub. Date: Jun. 22, 2006

(54) Methods for use of oral care compositions containing free-B-ring flavonoid anti-oxidants

(76) Inventors: Harsh M. Trivedi, Somerset, NJ (US); Tao Xu, East Brunswick, NJ (US)

Correspondence Address: COLGATE-PALMOLIVE COMPANY, 909 RIVER ROAD, PISCATAWAY, NJ, 08855, US

(21) Appl. No.: 11/280,668

(22) Filed: Nov. 16, 2005

Related U.S. Application Data
(60) Provisional application No. 60/639,329, filed on Dec. 22, 2004.

(51) Int. Cl. A61K 8/49

(52) U.S. Cl. 424/49

(57) ABSTRACT

The present invention relates to a method for providing an anti-oxidant to an oral cavity of a mammalian subject. An oral composition comprising an anti-oxidant active ingredient comprising a free-B-ring flavonoid and an orally acceptable carrier is contacted with oral tissue in the oral cavity. The anti-oxidant active ingredient reduces one or more reactive oxygen species, or free radicals in the oral cavity. Methods are also provided for preparing the anti-oxidant containing oral composition.

### CROSS REFERENCE TO RELATED APPLICATION

This application claims priority to U.S. Provisional Patent Application Ser. No. 60/639,329, filed Dec. 22, 2004, the contents of which are incorporated herein by reference.

### BACKGROUND OF THE INVENTION

Produce reactive oxygen species (ROS) are during various biochemical processes, and include superoxide anions, hydrogen peroxide, and hydroxyl radicals. The formation of ROS can occur as part of many cellular processes including mitochondrial respiration, immune cell responses, cell injury, heat, radiation of many origins, from metabolism of drugs and other chemicals. The ROS are highly reactive and modify important cellular macromolecules. ROS initiate or accelerate disease processes.

In one example, ROS are generated during inflammation by phagocytic leukocytes, such as activated neutrophils that produce an “oxidative burst” of superoxide radicals, which are believed to be an essential factor in producing the cytotoxic effect of activated neutrophils. Moreover, superoxide may be produced physiologically by endothelial cells for reaction with nitric oxide, a physiological regulator, forming peroxynitrite, ONOO which may decay and give rise to hydroxyl radical, -OH. Additional sources of oxyradicals are “leakage” of electrons from disrupted mitochondrial or endoplasmic reticular electron transport chains, prostaglandin synthesis, oxidation of catecholamines, and platelet activation.

ROS are thought to be involved in almost all disease processes and the ageing process. Increased ROS formation under pathological conditions is believed to cause cellular damage through the action of these highly reactive molecules by crosslinking proteins, mutagenizing DNA, and peroxidizing lipids.

Human periodontal diseases are inflammatory disorders that are the result of complex interactions between periodontopathogens and the host's immune response. It is believed that there are two interrelated aspects to the progression of periodontal disease, the first is the activation of the immune system of the host and the second is the production of oxygen radicals and their related metabolites. Increased production of oxygen radicals may contribute to oxidative stress, which is believed to be involved in periodontal disease.

Gingivitis is the inflammation or infection of the gums and the alveolar bones that support the teeth. Gingivitis is generally believed to be caused by bacteria in the mouth (particularly the bacteria instigated in plaque formation) and the toxins formed as byproducts from the bacteria. The toxins are believed to instigate oral tissue inflammation within the mouth. Periodontitis is a progressively worsened state of disease as compared to gingivitis, where the gums are inflamed and begin to recede from the teeth and pockets form therebetween, which ultimately may result in destruction of the bone and periodontal ligament. Thus, chronic infection and inflammation potentially results in the subsequent loss of teeth. Further, oral tissue inflammation can be caused by surgery, localized injury, trauma, or necrosis, or various systemic origins.

It is generally believed that the cellular components implicated by these diseases and conditions include epithelial tissue, gingival fibroblasts, and circulating leukocytes, all of which contribute to the host response to pathogenic factors generated by the bacteria. Thus, bacterial infection of the oral tissue ramps up the host's immune response and diminishes the healing process by generating free radical ROS species and up-regulating inflammatory mediators that cause significant tissue damage.

It would be desirable to have a method of treating a mammalian subject having cellular damage in oral tissue, in some circumstances caused by oral tissue inflammation, by reducing free radical reactive oxygen species to reduce the level of cellular damage to the oral tissue and to promote healing.

### BRIEF SUMMARY OF THE INVENTION

In various embodiments of the present invention, a method for reducing one or more free radical species in an oral cavity of a mammalian subject is provided. The method comprises contacting an oral composition comprising an anti-oxidant active ingredient and an orally acceptable carrier. The anti-oxidant active ingredient comprises at least one free-B-ring flavonoid and an orally acceptable carrier with an oral surface in the mammalian subject.

In other embodiments, a method for providing an anti-oxidant to an oral cavity of a mammalian subject is disclosed. The method comprises contacting oral tissue in the oral cavity of the mammalian subject, with an oral composition comprising an anti-oxidant active ingredient comprising a free-B-ring flavonoid and an orally acceptable carrier.

In yet other embodiments, a method of preparing an anti-oxidant oral composition is provided, where the method comprises mixing an anti-oxidant ingredient comprising at least one free-B-ring flavonoid with an orally acceptable oral composition carrier.

It has been discovered that compositions and methods of this invention impart advantages over the prior art oral compositions, by providing an oral care composition that is safe, stable, and highly effective as an anti-oxidant treatment for inflammation of oral tissue associated with such diseases as gingivitis and periodontitis. The anti-oxidant ingredient comprises compounds that are safe and derived from a natural botanical source, for example, Scutellaria baicalensis.

### DETAILED DESCRIPTION OF THE INVENTION

Various embodiments of the present invention provide a method of reducing one or more free radical species in an oral cavity of a mammalian subject. Such a method comprises contacting an oral composition comprising an anti-oxidant active ingredient and an orally acceptable carrier. The anti-oxidant active ingredient comprises at least one free-B-ring flavonoid and an orally acceptable carrier with an oral surface in the mammalian subject.

In other embodiments, a method for providing an anti-oxidant to an oral cavity of a mammalian subject comprises contacting oral tissue in the oral cavity of the mammalian subject, with an oral composition comprising an anti-oxidant active ingredient comprising a free-B-ring flavonoid and an orally acceptable carrier.

Contacting the oral tissue of a mammalian subject with the anti-oxidant in the oral composition serves to mitigate damage by free radical reactive oxygen species, and in an attendant mechanism can serve to reduce oral tissue inflammation by an alternate pathway than previously recognized (by minimizing damage from free radical species rather than the previously recognized suppression of pro-inflammatory cytokine production) in oral tissues. The method thereby reduces inflammation and promotes healing by reducing the damage from free radical species.

A “safe and effective amount” of an active ingredient in the various embodiments of the present invention refers to an amount that effects the targeted activity, (e.g., anti-oxidant activity at the target site of an host to be treated), without undue adverse side effects (such as toxicity, irritation, or allergic response), commensurate with a reasonable benefit/risk ratio when used in the manner of this invention. The specific “safe and effective amount” will vary with such factors as the particular condition being treated, the physical condition of the patient, the duration of treatment, the nature of concurrent therapy (if any), the specific dosage form to be used, the excipient employed, the solubility of the active ingredient therein, and the dosage regimen desired for the oral composition.

“Inflammation” of the oral tissue generally refers to a localized protective response elicited by injury or destruction of tissues, which serves to destroy, dilute, or sequester both the injurious agent and the injured tissue. In the acute form, it is characterized by pain, heat, redness, swelling, and loss of function. Chronic inflammation is a slow process and primarily characterized by the formation of new connective tissue. Chronic inflammation is often a continuation of acute inflammation or a prolonged low-grade form of inflammation (such as that associated with periodontitis or gingivitis) and usually causes permanent tissue damage. Histologically, inflammation involves a complex series of events, including dilation of arterioles, capillaries, and venules, with increased permeability and blood flow; exudation of fluids, including plasma proteins, and leukocytic migration into the inflammatory locus. Inflammation corresponds to enhanced levels of pro-inflammatory cellular mediators, as well as free radical species or reactive oxygen species (ROS), which are released from cells, for example, as the result of the interaction of an antigen with an antibody or by the action of antigen with a sensitized lymphocyte.

Sources of oral tissue inflammation include bacterial infection, surgery, localized injury, trauma or necrosis, or various systemic origins. Non limiting examples of oral diseases, conditions, and disorders associated with enhanced activity of cellular mediators of inflammation include gingivitis, periodontitis, exfoliation of teeth due to neutropenia, endodontic pathoses and its sequela, acute and chronic ulceration of the oral mucosa, acute necrotizing ulcerative gingivitis, dental caries, delayed wound healing, periodontal bone damage and acute and chronic osteomyelitis of the mandibular bone. In certain embodiments, the inflammatory disease or condition being treated is gingivitis or periodontitis.

Excessive concentrations of various forms of oxygen and of free radicals generated by inflamed tissue and the host's immune response can have serious adverse effects on living systems, including the peroxidation of membrane lipids, the hydroxylation of nucleic acid bases, and the oxidation of sulfhydryl groups and of other sensitive moieties in proteins. If uncontrolled, mutations and cellular death result.

Aerobic cells contains a number of defenses against the deleterious effects of oxyradicals and their reaction products. For example, biological antioxidants are present in a variety of cells, including enzymes, such as superoxide dismutase, catalase, selenium glutathione peroxidase, and phospholipid hydroperoxide glutathione peroxidase; and nonenzymatic biological antioxidants include tocopherols and tocotrienols, carotenoids, quinones, bilirubin, ascorbic acid, uric acid, and metal-binding proteins. Various antioxidants, being both lipid and water soluble, are found in all parts of cells and tissues, although each specific antioxidant often shows a characteristic distribution pattern. However, in the context of the oral cavity, there is mounting evidence that mammalian subjects suffering from various forms of gum disease have abnormally low levels of antioxidants (such as glutathione) in the surrounding oral tissue (e.g., in gingival crevicular fluids and the like), as where mammalian subjects with healthy gums have much higher levels of antioxidants.

While not limiting to the theory by which the present invention is bound, it is believed that certain flavonoid compounds prevent oxyradical-induced damage by scavenging free radical compounds (ROS) after they have been formed in the oral cavity. The present invention provides an anti-oxidant active ingredient for an oral composition that is safe for consumption, effective to remove dangerous oxyradicals, particularly superoxide and hydrogen peroxide, and is stable and efficacious within an oral composition that is relatively easy to manufacture.

In certain embodiments, the present invention is useful for preventing the development of diseases or to prevent cellular death and ageing of oral tissue. As used herein the term “prevention” pertains to a prophylactic treatment of an oral cavity of a mammalian subject, by contacting an oral composition comprising an anti-oxidant ingredient with oral tissue having a propensity for ageing, dying or becoming inflamed, diseased, or damaged.

In certain embodiments, a method is provided for treating diseases and disorders of the oral cavity and conditions associated with inflammation, infection and elevated levels of one or more reactive oxygen species. “Treating” involves the application of an oral composition comprising the anti-oxidant free-B-ring flavonoid after the development or physical manifestation of inflammatory response and free radical species generation, due to a disease or condition. Upon treating the inflamed tissue, the inflammation, disease, or condition is ameliorated or prevented from deteriorating to a worsened state by free radical destruction on the oral tissue. For example, the application of free-B-ring flavonoid anti-oxidant after the development of the inflammatory cascade comprises “treatment” of the disease or inflammatory/infectious symptoms.

In certain embodiments, the method of treatment comprises administering a therapeutically beneficial amount of the oral composition comprising an anti-oxidant comprising free-B-ring flavonoid at repeated intervals over a period time, from one week up to a lifetime. For example, a typical method for treating diseases, conditions, and disorders of the oral cavity, as well as overall preventative treatment to slow the ageing process, comprises administration of a therapeutically beneficial amount of an oral composition comprising free-B-ring flavonoid anti-oxidant, administered on a daily basis.

In various embodiments, application or contacting can be accomplished by rinsing, coating, brushing, or layering using appropriate dressing materials. In various embodiments, application of the composition comprises the use of an application device which aids in maintaining the contact time of the anti-oxidant active ingredient comprising the free-B-ring flavonoid anti-oxidant to the target tissue for a sufficient time as to allow the pharmacological inhibition or reduction of reactive oxygen species within and near the oral tissue.

In various embodiments of the present invention, the oral composition comprises an anti-oxidant active ingredient that reduces one or more free radical or reactive oxygen species. In preferred embodiments of the present invention, the oral composition comprises an anti-oxidant that is a free-B-ring flavonoid.

The present invention provides oral care compositions and methods for administration or application to, or use with, a human or other animal subject. As referred to herein, an “oral care composition” is any composition that is suitable for administration or application to the oral cavity of a human or animal subject for enhancing the health, hygiene or appearance of the subject, preferably providing such benefits as: the prevention or treatment of a condition or disorder of the teeth, gums, mucosa or other hard or soft tissue of the oral cavity; the prevention or treatment of a systemic condition or disorder; the provision of sensory, decorative or cosmetic benefits; and combinations thereof. In various preferred embodiments, an oral care composition is not intentionally swallowed, but is rather retained in the oral cavity for a time sufficient to effect the intended utility. Preferably, specific materials and compositions to be used in this invention are, accordingly, pharmaceutically- or cosmetically-acceptable. As used herein, such a “pharmaceutically acceptable” or “cosmetically acceptable” component is one that is suitable for use with humans and/or animals to provide the desired therapeutic, prophylactic, sensory, decorative, or cosmetic benefit without undue adverse side effects (such as toxicity, irritation, and allergic response) commensurate with a reasonable benefit/risk ratio.

In preferred embodiments, the anti-oxidant ingredient of the oral compositions of the present invention comprises at least one free-B-ring flavonoid. Flavonoids are a group of compounds that have the same general structure and are found in higher plants. The term flavonoids includes such classes of compounds as flavones, flavans, flavonols, dihydroflanonols, flavonones, and derivatives thereof.

In various embodiments of the present invention, the anti-oxidant active ingredient comprises a free-B-ring flavonoid, which refers to a flavonoid compound that lacks any substituent groups on the aromatic “B” ring, as shown in the following structure.

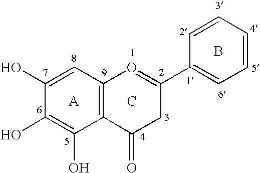

Free-B-ring flavonoids constitute a group of flavonoids that generally contain a 2,3-double bond and/or a 4-oxo group, but have no substitute groups on the aromatic B ring. Free-B-ring flavonoids are relatively rare and can be isolated from a variety of different plant parts, including, but not limited to, stems, stem barks, trunks, trunk barks, twigs, tubers, roots, root barks, young shoots, tissues, seeds, rhizomes, flowers, and other reproductive organs, leaves and other aerial parts. In various embodiments, free-B-ring flavonoids are isolated from plants of the family Lamiaceae. In various embodiments, the free-B-ring flavonoids are isolated from plants of the subfamily Scutellarioideae. In various embodiments, the free-B-ring flavonoids are isolated from plants of the genus Scutellaria. In certain embodiments, the free-B-ring flavonoids used as anti-oxidants in oral care compositions of the present invention are isolated from plants of the species Scutellaria baicalensis. The Chinese medicinal plant Scutellaria baicalensis contains significant amounts of free-B-ring flavonoids, including baicalein, baicalin, wogonin, and baicalenoside. The term “free-B-ring flavonoids” as used herein, also encompasses synthetic or semi-synthetic equivalents of such natural extracts or active components thereof. Compositions comprising free-B-ring flavonoids have previously been shown to inhibit general activity of the cyclooxygenase enzyme COX-2, however, have not previously been recognized for their anti-oxidant properties for use in oral care compositions. In preferred embodiments, the anti-oxidant active ingredient comprises either of two particularly efficacious and useful flavonoids isolated from S. baicalensis: 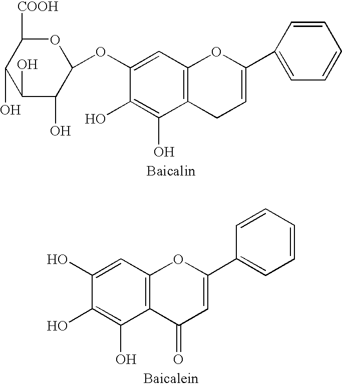
Baicalin (also known by the Chinese name “Huangqingan”) is 5,6-Dihydroxyflavone-7-O-glucoside, and Baicalein (also known by the Chinese name “Huangqinsu”) is 5,6,7-Trihydroxyflavone. In various embodiments, the anti-oxidant active ingredient of the oral compositions of the present invention may comprise baicalin, baicalein, or mixtures thereof.

Anti-oxidant active ingredients, such as the free-B-ring flavonoids of the present invention, are capable of reducing or neutralizing free radical compounds, including reactive oxygen species (ROS) such as superoxide anions, hydrogen peroxide, and hydroxyl anions. In certain embodiments, the predicted efficacy of an anti-oxidant active ingredient in an oral composition in vivo can be measured by the composition's ability to significantly reduce the oxidation of lipid peroxidases in vitro. The level of lipid peroxides (LPO) is an index of cellular membrane damage caused by the action of free radicals. For example, the membranes of the organelles within the cells (mitochondria, lysosomes, and peroxisomes) can be damaged. Membrane proteins, membrane lipids and cholesterol can be damaged due to an insufficiency of antioxidants to deal with the level of oxidative stress/free radicals. The elevation of LPOs serves as an indication of high free radical activity and an early warning of the potential long-term effects of oxidative stress. As previously described above, the outcome of long-term oxidative stress is chronic degenerative disease. Thus, a significant reduction of LPO versus a control is indicative of anti-oxidant activity. Such a measurement can be conducted by a commercially available assay, such as LPO-CC Kamiya Biomedical Kit, for example. In particular, it is important that the anti-oxidant oral composition is formulated in a stable formulation that demonstrates anti-oxidant efficacy of the active ingredient in the oral composition, notwithstanding the efficacy of the compound by itself.

Additionally, the concentration of antioxidant ingredient in the oral care composition depends upon the relative concentration of the active compounds in an extract or the purity of such ingredients, and may vary as recognized by one of skill in the art. Additionally, the concentration of the active ingredients is typically dependent upon the form of the oral composition. For example, mouthrinses typically have a relatively low concentration of an active ingredient, as where dentifrices, gels, or toothpowders have a higher concentration to achieve the same delivered dosage based on ease of dispersion. Likewise, confectionary compositions typically have a relatively wide range of concentrations of active ingredient to enable sufficient dispersion as they dissolve or are masticated.

In various embodiments of the present invention, the anti-oxidant active ingredient is present in the oral composition at a concentration of from about 0.001 to about 10%. In other embodiments, the anti-oxidant active ingredient is present from about 0.01 to about 5%. In certain embodiments, the anti-oxidant active ingredient is present from about 0.1 to about 3%. In other embodiments, the anti-oxidant active ingredient is present from about 0.1 to about 1%. In certain embodiments, the anti-oxidant active ingredient is selected from the group of free-B-ring flavonoids (flavones) selected from the group consisting of: baicalin, baicalein, or mixtures thereof. In an embodiment where the anti-oxidant comprises baicalin, it is preferably present at a concentration of greater than about 0.2%. In other embodiments, where the anti-oxidant active ingredient comprises baicalein, the baicalein is present at greater than about 0.1%. Certain embodiments of the present invention comprise mixtures of baicalin and baicalein as the active ingredient, where the free-B-ring flavonoid compounds are present at greater than 1%.

In one embodiment, the free-B-ring flavonoids are isolated from Scutellaria baicalensis by extraction from the dried root using an appropriate solvent. Preferred solvents include methanol, ethanol, methylene chloride, hexane, cyclohexane, pentane, petroleum ether, chloroform, hydrochloric acid, ethylene dichloride, methanol:THF, and hydrofluoroalkanes, such as 1,1,1,2-tetrafluoroethane or HFA-13A. Generally, one part of plant tissue (dry basis) is extracted with from about 5 to about 50 parts, preferably from about 15 parts to about 30 parts of solvent using an extraction apparatus where the solvent is contacted with the bark to obtain a concentrated paste which is then subjected to one or more additional extraction steps with different solvents to further concentrate the originally obtained paste over an extended period of time, preferably from about 6 hours to about 1-2 days, more preferably for about 1 day. In preferred embodiments, the natural extract active ingredients used in oral care compositions are of reproducible, stable, and have microbiological safety. In one embodiment of the present invention, the extract is isolated by supercritical fluid extraction (SFE) using carbon dioxide (CO₂) or by steam distillation, as recognized by one of skill in the art. It should be noted that other botanical sources of free-B-ring flavonoids, and in particular the preferred baicalin or baicalein compounds, are also suitable for use with the present invention.

A suitable vehicle or carrier includes one or more compatible solid or liquid fillers, diluents, excipients, or encapsulating substances, which are suitable for topical administration to oral tissue surfaces. It is preferred that the orally acceptable carrier does not cause irritation, swelling or pain and does not typically produce an allergic or untoward reaction such as gastric upset, nausea or dizziness. Selection of specific carrier components is dependant on the desired product form, including dentifrices, toothpastes, tooth powders, prophylaxis pastes, mouth rinses, lozenges, gums, gels, paints, animal products, and the like.

In various embodiments, such as for toothpastes, creams and gels, the oral composition contains a natural or synthetic thickener or gelling agent, which other than silica thickeners, include natural and synthetic gums and colloids. Such suitable thickeners include naturally occurring polymers such as carrageenans, xanthan gum, synthetic thickener such as polyglycols of varying molecular weights sold under the tradename Polyox and cellulose polymers such as hydroxyethyl cellulose and hydroxpropyl cellulose. Other inorganic thickeners include natural and synthetic clays such as hectorite clays, lithium magnesium silicate (laponite) and magnesium aluminum silicate (Veegum). Other suitable thickeners are synthetic hectorite, a synthetic colloidal magnesium alkali metal silicate complex clay available for example as Laponite (e.g., CP, SP 2002, or D) marketed by Laporte Industries Limited. Laponite D analysis shows, approximately, 58.00% SiO₂, 25.40% MgO, 3.05% Na₂O, 0.98% Li₂O, and some water and trace metals, and has a true specific gravity of 2.53 and an apparent bulk density (g/mL at 8% moisture) of 1.0. In certain embodiments, the thickening agent is present in the dentifrice composition in amounts of about 0.1 to about 10%, preferably about 0.5 to about 5.0%.

Other suitable thickeners include Irish moss, gum tragacanth, starch, polyvinylpyrrolidone, hydroxyethyl propyl cellulose, hydroxybutyl methyl cellulose, hydroxypropyl methyl cellulose, hydroxyethyl cellulose (e.g., available as NATROSOLI), sodium carboxymethyl cellulose, and colloidal silica such as finely ground SYLOID (e.g., 244).

Various embodiments of the present invention also comprise a surface active agent, which may function as a surfactant, emulsifier, and/or foam modulator. Surface active agents generally achieve increased prophylactic action, by thoroughly dispersing the active ingredients throughout the oral cavity. Suitable emulsifying agents are those which are reasonably stable and foam throughout a wide pH range, including non-soap anionic, nonionic, zwitterionic and amphoteric organic synthetic detergents. Further, surface active ingredients preferably render the instant compositions more cosmetically acceptable. The organic surface-active material is preferably anionic, nonionic or ampholytic in nature, and preferably a detersive material which imparts to the composition detersive and foaming properties. In certain embodiments, one or more surfactants are present in the oral composition of the present invention in the range from about 0.001% to about 5%, preferably from about 0.5% to about 2.5%.

Nonionic surfactants useful in the compositions of the present invention include compounds produced by the condensation of alkylene oxides (especially ethylene oxide) with an organic hydrophobic compound, which may be aliphatic or alkylaromatic in nature. One group of surfactants is known as “ethoxamers”—they are condensation products of ethylene oxide with fatty acids, fatty alcohols, fatty amides, polyhydric alcohols, (e.g., sorbitan monostearate) and the like. “Polysorbates” is the name given to a class of nonionic surfactants prepared by ethoxylating the free hydroxyls of sorbitan-fatty acid esters. They are commercially available, for example as the TWEEN® surfactants of ICI. Non-limiting examples include Polysorbate 20 (polyoxyethylene 20 sorbitan monolaurate, Tween® 20) and Polysorbate 80 (polyoxyethylene 20 sorbitan mono-oleate, TWEEN® 80). Preferred polysorbates include those with about 20 to 60 moles of ethylene oxide per mole of sorbitan ester.

Other suitable nonionic surfactants include poly(oxyethylene)-poly(oxypropylene) block copolymers, especially triblock polymers of this type with two blocks of poly(oxyethylene) and one block of poly(oxypropylene). Such copolymers are known commercially by the non-proprietary name of poloxamers, the name being used in conjunction with a numeric suffix to designate the individual identification of each copolymer. Poloxamers may have varying contents of ethylene oxide and propylene oxide, leading to a wide range of chemical structures and molecular weights. One preferred poloxamer is Poloxamer 407. It is widely available, for example under the tradename PLURONIC® F127 of BASF Corporation.

Other non-limiting examples of suitable nonionic surfactants include products derived from the condensation of ethylene oxide with the reaction product of propylene oxide and ethylene diamine, long chain tertiary amine oxides, long chain tertiary phosphine oxides, long chain dialkyl sulfoxides and the like.

Other surfactants useful in various embodiments of the present invention include zwitterionic synthetic surfactants. Certain of these can be broadly described as derivatives of aliphatic quaternary ammonium, phosphonium, and sulfonium compounds, in which the aliphatic radicals can be straight chain or branched, and where one of the aliphatic substituents contains from 8 to 18 carbon atoms and one contains an anionic water-solubilizing group, e.g., carboxy, sulfonate, sulfate, phosphate or phosphonate. One example of a suitable zwitterionic surfactant is 4-(N,N-di(2-hydroxyethyl)-N-octadecylammonio)-butane-1-carboxylate.

Other suitable zwitterionic surfactants include betaine surfactants, such as those disclosed in U.S. Pat. No. 5,180,577, the contents of which are incorporated herein by reference. Typical alkyldimethyl betaines include decyl betaine 2-(N-decyl-N,N-dimethylammonio) acetate, cocobetaine, myristyl betaine, palmityl betaine, lauryl betaine, cetyl betaine, stearyl betaine, and the like. The amidobetaines are exemplified by cocoamidoethyl betaine, cocoamidopropyl betaine, lauramidopropyl betaine and the like. Particularly useful betaine surfactants include cocoamidopropyl betaine and lauramido propyl betaine.

Suitable examples of anionic surfactants are water-soluble salts of higher fatty acid monoglyceride monosulfates, such as the sodium salt of the monosulfated monoglyceride of hydrogenated coconut oil fatty acids, higher alkyl sulfates such as sodium lauryl sulfate (SLS), alkyl aryl sulfonates such as sodium dodecyl benzene sulfonate, higher alkyl sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine which are preferably substantially free from soap or similar higher fatty acid material.

In various embodiments of the present invention, where the carrier of the oral care composition is solid or a paste, the oral composition preferably comprises a dentally acceptable abrasive material, which may serve to either polish the tooth enamel or provide a whitening effect.

In the preparation of a dentifrice composition, abrasives, which may be used in the practice of the present invention, include silica abrasives such as precipitated silicas having a mean particle size of up to about 20 microns, such as Zeodent 115, marketed by J. M. Huber. One useful abrasive is marketed under the trade designation Zeodent 105 by J. M Huber Co, which has a low abrasiveness to tooth enamel, and is a precipitated silica that is about 7 to about 10 microns in diameter, has a BET surface area of 390 m²/g of silica, and an oil absorption of less than 70 cm³/100 g of silica. Other useful dentifrice abrasives include sodium metaphosphate, potassium metaphosphate, tricalcium phosphate, dihydrated dicalcium phosphate, anhydrous dicalcium phosphate, aluminum silicate, calcined alumina, bentonite or other siliceous materials, or combinations thereof.

In other embodiments of the present invention, useful abrasive materials for preparing dentifrice compositions include silica gels and precipitated amorphous silica having an oil absorption value of less than 100 cm³/100 g silica and preferably in the range of from about 45 cm³/100 g to less than about 70 cm³/100 g silica. Oil absorption values are measured using the ASTA Rub-Out Method D281. These silicas are colloidal particles having an average particle size ranging from about 3 microns to about 12 microns, and more preferably between about 5 to about 10 microns and a pH range from 4 to 10, preferably 6 to 9 when measured as a 5% slurry. One useful abrasive is marketed under the trade designation Zeodent 105 by J. M Huber Co, which has a low abrasiveness to tooth enamel, and is a precipitated silica that is about 7 to about 10 microns in diameter, has a BET surface area of 390 m²/g of silica, and an oil absorption of less than 70 cm³/100 g of silica. Further suitable abrasives useful with various embodiments of the present invention are low oil of absorption silica abrasives such as those marketed under the trade designation Sylodent XWA or Sylodent 783 by Davison Chemical Division of W. R. Grace & Co., Baltimore, Md. 21203. Sylodent XWA 650, a silica hydrogel composed of particles of colloidal silica having a water content of 29% averaging from about 7 to about 10 microns in diameter, and an oil absorption of less than 70 cm³/100 g of silica is a preferred example of a low oil absorption silica abrasive useful in the practice of the present invention. The abrasive is present in the dentifrice composition of the present invention at a concentration of about 10 to about 40% and preferably about 15 to about 30%.

Other suitable polishing materials include the particulate thermosetting resins, such as melamine, phenolic, and urea-formaldehydes, and cross-linked polyepoxides and polyesters. Preferred polishing materials include crystalline silica having particle sizes of up to about 5 microns, a mean particle size of up to about 1.1 microns, and a surface area of up to about 50,000 cm²/g, silica gel or colloidal silica, and complex amorphous alkali metal aluminosilicate.

Particularly preferred abrasives in accordance with certain embodiments of the present invention comprise dihydrated dicalcium phosphate, anhydrous dicalcium phosphate, precipitated calcium carbonate or combinations thereof.

In embodiments where the dentifrice is a clear or transparent gel, a polishing agent of colloidal silica, such as those sold under the trademark SYLOID as Syloid 72 and Syloid 74 or under the trademark SANTOCEL as Santocel 100 alkali metal almuino-silicate complexes are particularly useful, since they have refractive indices close to the refractive indices of gelling agent-liquid (including water and/or humectant) systems commonly used in dentifrices.

In certain embodiments, the abrasives may also include whiteness-imparting abrasive particles which include for example, a metal oxide. The metal oxide can comprise any metal oxide that provides a white color, such as, for example, titanium oxide, aluminum oxide, tin oxide, calcium oxide, magnesium oxide, barium oxide, or a combination thereof. Certain whiteness imparting abrasives are also pearlescent particles, which comprise a single mineral or chemical species, such as, for example a silicate such as mica, or bismuth oxychloride. By “mica” it is meant any one of a group of hydrous aluminum silicate minerals with platy morphology and perfect basal (micaceous) cleavage. Mica can be, for example, sheet mica, scrap mica or flake mica, as exemplified by muscovite, biotite or phlogopite type micas. In some embodiments, the pearlescent particles can comprise a complex comprising more than one mineral or chemical species, such as, for example, mica coated with a metal oxide such as titanium oxide.

In embodiments where the dentrifrice is in a solid or paste form, the abrasive material is generally present at about 10% to about 99% of the oral composition. In certain embodiments, the polishing material is present in amounts ranging from about 10% to about 75% in toothpaste, and from about 70% to about 99% in toothpowder.

In various embodiments of the present invention, water is also present in the oral composition, as referred to above. Water employed in the preparation of commercially suitable toothpastes, gels, and mouthwashes should preferably be deionized, ultraviolet treated, and free of organic impurities. Water generally comprises from about 10% to 50%, preferably from about 20% to 40%, of the toothpaste compositions herein. The water is free water which is added, plus that which is introduced with other materials for example, such as that added with sorbitol.

In various embodiments, the oral care composition of the present invention contains a flavoring agent. Flavoring agents which are used in the practice of the present invention include essential oils as well as various flavoring aldehydes, esters, alcohols, and similar materials. Any suitable flavoring or sweetening material may also be employed. Examples of suitable flavoring constituents are flavoring oils, e.g. oil of spearmint, peppermint, wintergreen, sassafras, clove, sage, eucalyptus, marjoram, cinnamon, lemon, lime, orange, grapefruit, and methyl salicylate. Also useful are such chemicals as menthol, carvone, and anethole. Suitable sweetening agents include sucrose, lactose, maltose, sorbitol, xylitol, sodium cyclamate, perillartine, AMP (aspartyl phenyl alanine, methyl ester), saccharine and the like. The flavor and sweetening agents may each or together be incorporated into the oral composition at a concentration of about 0.001 to about 5% and preferably about 0.5 to about 2%.

The composition of the invention can be incorporated into chew articles or toys that are formed in a variety of designs and sizes, as known to those of skill in the art, and preferably provide some level of physical interaction with the tooth and gum surface, promoting gingival stimulation and/or sub-gingival particle release. Examples of such toys can be bones, balls, and ropes. Further, it is preferred that the chew toys are capable of carrying active ingredients, either through an internal reservoir, by impregnation into the material, or coating onto a surface of the toy, for example. Chew articles of the present embodiment are preferably formed of a non-toxic edible material, including by way of example, rawhide or polymers such as polyester or polyisoprene.

Food products and supplements for animals are well known in the art and are preferably made with any suitable dough. Food supplement dough generally comprises at least one flour, meal, fat, water, and optionally particulate proteinaceous particles (for texturization) and flavor. For instance, when the desired product is a biscuit, a conventional dough can be used, optionally containing discrete particles of meat and/or meat byproducts or farinaceous material. Examples of suitable dough for the production of hard and soft (including humectant for water control) animal biscuits are disclosed in U.S. Patent. Nos. 5,405,836 to Richar, et al.; 5,000,943 to Scaglione, et al.; 4,454,163 and 4,454,164 both to Gellman, et al. Such compositions are preferably baked. The active ingredient may be added with the flavor, included in an interior reservoir with a soft center, or coated onto the surface of a baked food supplement by dipping or spraying. Any other suitable means known to one of skill in the art for delivering active ingredients to animals are also contemplated by the present invention.

The compositions used in accordance with the present invention optionally comprise an optional active material aside from the anti-oxidant free-B-ring flavonoid active ingredient. Such additional oral care active ingredients are operable for the prevention or treatment of a condition or disorder of hard or soft tissue of the oral cavity, the prevention or treatment of a physiological disorder or condition, or to provide a cosmetic benefit. In such embodiments, the one or more additional active ingredients do not inhibit the efficacy of the anti-oxidant ingredient described above.

In various embodiments, the active is a “systemic active” which is operable to treat or prevent a disorder which, in whole or in part, is not a disorder of the oral cavity. In various embodiments, the active is an “oral care active” operable to treat or prevent a disorder or provide a cosmetic benefit within the oral cavity (e.g., to the teeth, gingiva or other hard or soft tissue of the oral cavity). Optional oral care actives among those useful herein include anti-tartar agents, antibacterial agents, anti-inflammatory agents, anticaries agents, whitening agents, densensitizing agents, vitamins, compatible enzymes, chlorophyll compounds, periodontal actives, breath freshening agents, malodour control agents, salivary stimulants and combinations thereof, which are well known to one of skill in the art. It is understood that while general attributes of each of the above categories of actives may differ, there may some common attributes and any given material may serve multiple purposes within two or more of such categories of actives.

Compositions of the present invention may also be used for the treatment or prevention of systemic disorders, such as the improvement of overall systemic health characterized by a reduction in risk of development of systemic diseases, such as cardiovascular disease, stroke, diabetes, severe respiratory infection, premature and low birth weight infants (including associated post-partum dysfunction in neurologic/developmental function), and associated increased risk of mortality. Such methods and additional active ingredients useful for treating such conditions include those disclosed in U.S. Patent Publication 2003/0206874, Doyle et al., published Nov. 6, 2003. Actives among those useful herein are also disclosed in U.S. Pat. No. 6,290,933, Durga et al., issued Sep. 18, 2001 and U.S. Pat. No. 6,685,921, Lawlor, issued Feb. 3, 2004. Actives useful herein are optionally present in the compositions of the present invention in safe and effective amounts.

The oral composition of the present invention may contain an anticaries agent, such as a fluoride ion source or a fluorine-providing component. In various embodiments, the fluoride based anticaries agent in present in an amount sufficient to supply about 25 ppm to 5,000 ppm of fluoride ions. Useful anticaries agents include inorganic fluoride salts, such as soluble alkali metal salts. For example, preferred fluoride sources useful in the oral composition are sodium fluoride, potassium fluoride, sodium fluorosilicate, ammonium fluorosilicate, sodium monfluorophosphate (MFP), and amine fluorides, including olaflur (N′-octadecyltrimethylendiamine-N,N,N′-tris (2-ethanol)-dihydrofluoride). Tin based compounds, including stannous fluoride and stannous chloride are also useful herein. In certain embodiments, sodium fluoride or MFP is preferred as an anticaries ingredient.

In various embodiments, the oral compositions of the present invention comprise antitartar agents to prevent and/or minimize calculus formation. One or more of such agents can be present.

Suitable anticalculus agents include without limitation: phosphates and polyphosphates. Phosphate and polyphosphate salts are generally employed in the form of their wholly or partially neutralized water soluble cationic species (e.g., potassium, sodium or ammonium salts, and any mixtures thereof). Thus, useful inorganic phosphate and polyphosphate salts illustratively include monovalent cations with monobasic, dibasic and tribasic phosphates; tripolyphosphate and tetrapolyphosphate; mono-, di-, tri- and tetra-pyrophosphates; and cyclophosphates (also generally known in the art as “metaphosphates”). Useful monovalent cations of such phosphate salts include hydrogen, monovalent metals including alkali metals, and ammonium, for example.

Examples of useful antitartar agents include Na₅P₃O₁₀ (sodium tripolyphosphate or STPP), tetraalkali metal pyrophosphate salts such as Na4P₂O₇ (tetrasodium pyrophosphate or TSPP), K4P207 (tetrapotassium pyrophosphate), Na₂K₂P₂O₇ (disodium dipotassium pyrophosphate), Na₂H₂P₂O₇ (disodium dihydrogen pyrophosphate) and K₂H₂P₂O₇ (dipotassium dihydrogen pyrophosphate). Cyclophosphates, which are generally referred to as “metaphosphates”, are cyclic phosphate anion compounds. Those useful as tartar control agents include, sodium hexametaphosphate and sodium trimetaphosphate, for example. In one embodiment, the active anticalculus system comprises sodium tripolyphosphate (STPP) and/or tetrasodium pyrophosphate (TSPP).

Other suitable tartar control agents include polyaminopropanesulfonic acid (AMPS), zinc citrate trihydrate, polypeptides such as polyaspartic and polyglutamic acids, polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts.

In various embodiments where the anticalculus/anti-tartar active ingredients are present in the oral compositions, they range in concentration from about 0.01 to about 10% by weight, more preferably between about 1 to about 5% by weight.

Additionally, various embodiments of the present invention include an anticalculus system that further comprises a synthetic anionic linear polycarboxylate polymer. The anionic linear polycarboxylate is generally synthesized by using an olefinically or ethylenically unsaturated carboxylic acid that contains an activated carbon-to-carbon olefinic double bond and at least one carboxyl group. The acid contains an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrilacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other olefinic monomers copolymerizable with such carboxylic monomers include vinyl acetate, vinyl chloride, dimethyl maleate and the like. The synthetic anionic linear polymeric polycarboxylate component is mainly a hydrocarbon with optional halogen and O-containing substituents and linkages as present in for example ester, ether and OH groups. The copolymers preferably contain sufficient carboxylic salt groups for water-solubility. The terms “synthetic” and “linear”do not include known thickening or gelling agents comprising carboxymethylcellulose and other derivatives of cellulose and natural gums, nor Carbopols having reduced solubility due to cross-linkages.

Preferred are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 2,500,000. These copolymers are commercially available, for example as Gantrez AN-139 (M.W. 1,100,000), AN-119 (M.W. 200,000) and S-97 Solution (M.W. 1,500,000), from ISP Corporation.

In various embodiments, where the anti-tartar/anticalculus system comprises a synthetic anionic polycarboxylate, it is preferably present from about 0.001 to about 5 weight %. In another embodiment, the synthetic anionic polycarboxylate is present from about 0.5 to about 1.5 weight %, most preferably at about 1 weight % of the oral care composition. In one embodiment according to the present invention, the anticalculus system comprises a copolymer of maleic anhydride and methyl vinyl ether, such as for example, the Gantrez S-97 product discussed above. In one embodiment, the antitartar active ingredient system of the oral care composition comprises TSPP at about 0.5 to about 1.5% by weight, STPP at about 1 to about 10% by weight, and a copolymer of maleic anhydride and methyl vinyl ether at about 0.5 to about 1.5% by weight.

Synthetic anionic polycarboxylates may also be used in the dentifrice compositions of the present invention as an efficacy enhancing agent for certain active ingredients, including antibacterial, antitartar (as discussed above) or other active agents within the oral composition. Such anionic polycarboxylates are generally employed in the form of their free acids or preferably partially or more preferably fully neutralized water soluble alkali metal (e.g. potassium and preferably sodium) or ammonium salts. As discussed above, preferred copolymers are of maleic anhydride or acid with another polymerizable ethylenically unsaturated monomer, preferably methylvinylether/maleic anhydride having an approximate molecular weight (M.W.) of about 30,000 to about 2,500,000 most preferably about 30,000 to about 2,000,000. Examples of these copolymers are available from ISP corporation under the tradename Gantrez, e.g. AN 139 (M.W. 1,100,000), AN 119 (M.W. 200,000); S-97 Pharmaceutical Grade (M.W. 1,500,000), AN 169 (M.W. 2,000,000), and AN 179 (M.W. 2,400,000); wherein the preferred copolymer is S-97 Pharmaceutical Grade (M.W. 1,500,000).

The anionic polycarboxylate, is employed in certain embodiments in amounts effective to achieve the desired enhancement of the efficacy of any antibacterial, antitartar or other active agent within the dentifrice composition. Generally, the anionic polycarboxylates is present within the dentifrice composition from about 0.05% to about 5% by weight, preferably from about 0.5% to about 2.5% by weight.

Various optional oral care actives may be included in the oral composition of the present invention including those described above, such as antibacterial agents, antiplaque agents, anti-adhesion (that prevent adhesion of plaque to an enamel surface), anti-oxidant (such as Vitamin E or coenzyme Q10), anticaries agents, densensitizing agents (such as potassium citrate, potassium tartrate, potassium chloride, potassium sulfate and potassium nitrate), whitening agents (such as, urea peroxide, sodium percarbonate, sodium perborate and polyvinylpyrrolidone-H₂O₂); compatible enzymes; anti-inflammatory agents (such as, steroidal agents including flucinolone and hydrocortisone, and nonsteroidal agents (NSAIDs)), tartar control agents, periodontal actives, chlorophyll compounds, nutrients (such as vitamins, minerals, and amino acids, lipotropics, fish oil, coenzymes and the like) abrasives, breath freshening/malodour control agents (such as zinc salts such as zinc gluconate, zinc citrate, zinc chlorite, and α-ionone), and salivary stimulants (such as such as citric, lactic, malic, succinic, ascorbic, adipic, fumaric and tartaric acids); and any other suitable ingredients for oral care known to one of skill in the art. These additives, when present, are incorporated in the dentifrice composition in amounts that do not substantially adversely affect the properties and characteristics desired, generally from concentrations of about 0.001 to about 10%.

Various other materials may be incorporated in oral compositions of this invention including preservatives, such as sodium benzoate, and silicones, for example. These adjuvants, when present, are incorporated in the compositions in amounts which do not substantially adversely affect the properties and characteristics desired.

### EXAMPLE I

A dentifrice composition having the ingredients listed in Table I is prepared by the following method. The baicalin is isolated from an extract of S. baicalensis.

Sodium saccharin, sodium monofluorophosphate, TSPP, and any other salts are dispersed in water and mixed in a conventional mixer under agitation. The humectants e.g., glycerin and sorbitol, are added to the water mixture under agitation. Then organic thickeners, such as carageenan, and any polymers, are added. The resultant mixture is agitated until a homogeneous gel phase is formed. The mixture is then transferred to a high-speed vacuum mixer; where the dicalcium phosphate abrasives are added. The mixture is then mixed at high speed for from 5 to 30 minutes, under vacuum of from about 20 to 50 mm of Hg, preferably about 30 mm Hg. The flavor oil is weighed out and baicalin is then added to the flavor oil. The flavor oil and flavonoid mixture is added to the mixture. Lastly, surfactants, such as sodium lauryl sulfate (SLS) are charged into the mixer. The resultant product is a homogeneous, semi-solid, extrudable paste or gel product.

TABLE I

| Ingredient | Final Wt. % |
|---|---|
| Baicalin | 0.6 |
| Sorbitol | 14.0 |
| Glycerin | 10.8 |
| Sodium monofluorophosphate | 0.8 |
| Sodium saccharin | 0.1 |
| Tetra Sodium Pyrophosphate (TSPP) | 0.3 |
| Carrageenan (Viscarin) | 0.9 |
| Dicalcium Phosphate Anhydrous | 3.9 |
| Dicalcium Phosphate Dihydrate | 45.2 |
| Sodium lauryl sulfate | 1.5 |
| Flavor | 1.0 |
| Blue Color Solution | 0.05 |
| De-ionized and UV treated Water | Q.S. |

### EXAMPLE II

A dentifrice composition having the ingredients listed in Table II is prepared by the following method. The baicalein is isolated from an extract of S. baicalensis. Sodium saccharin, sodium monofluorophosphate, TSPP, and any other salts are dispersed in water and mixed in a conventional mixer under agitation. The humectants e.g., glycerin and sorbitol, are added to the water mixture under agitation. Then organic thickeners, such as carageenan, are added. The resultant mixture is agitated until a homogeneous gel phase is formed. The mixture is then transferred to a high-speed vacuum mixer; where the dicalcium phosphate abrasives are added. The mixture is then mixed at high speed for from 5 to 30 minutes, under vacuum of from about 20 to 50 mm of Hg, preferably about 30 mm Hg.

The flavor oil is weighed out and baicalein is then added to the favor oil. The flavor oil and flavonoid mixture is added to the mixture. Sodium lauryl sulfate (SLS) is then added into the mixer. The resultant product is a homogeneous, semi-solid, extrudable paste or gel product.

TABLE II

| Ingredient | Final Wt. % |
|---|---|
| Baicalein | 0.2 |
| Sorbitol | 14.0 |
| Glycerin | 10.8 |
| Sodium monofluorophosphate | 0.8 |
| Sodium saccharin | 0.1 |
| Tetra Sodium Pyrophosphate (TSPP) | 0.3 |
| Carrageenan (Viscarin) | 0.9 |
| Dicalcium Phosphate Anhydrous | 3.9 |
| Dicalcium Phosphate Dihydrate | 45.2 |
| Sodium lauryl sulfate | 1.5 |
| Flavor | 1.0 |
| Blue Color Solution | 0.05 |
| De-ionized and UV treated Water | Q.S. |

### EXAMPLE III

A dentifrice composition having the ingredients listed in Table III is prepared by the following method. The baicalein and baicalin are isolated from an extract of S. baicalensis. Sodium saccharin, sodium monofluorophosphate, TSPP, and any other salts are dispersed in water and mixed in a conventional mixer under agitation. The humectants e.g., glycerin and sorbitol, and polymers are added to the water mixture under agitation. Then organic thickeners, such as carageenan, are added. The resultant mixture is agitated until a homogeneous gel phase is formed. The mixture is then transferred to a high-speed vacuum mixer; where the dicalcium phosphate abrasives are added. The mixture is then mixed at high speed for from 5 to 30 minutes, under vacuum of from about 20 to 50 mm of Hg, preferably about 30 mm Hg.

The flavor oil is weighed out and baicalin/baicalein is then added to the flavor oil. The flavor oil and flavonoid mixture is added. Sodium lauryl sulfate (SLS) is then added into the mixer. The resultant product is a homogeneous, semi-solid, extrudable paste or gel product.

TABLE III

| Ingredient | Final Wt. % |
|---|---|
| Baicalein | 0.2 |
| Baicalin | 0.6 |
| Sorbitol | 14.0 |
| Glycerin | 10.8 |
| Sodium monofluorophosphate | 0.8 |
| Sodium saccharin | 0.1 |
| Tetra Sodium Pyrophosphate (TSPP) | 0.3 |
| Carrageenan (Viscarin) | 0.9 |
| Dicalcium Phosphate Anhydrous | 3.9 |
| Dicalcium Phosphate Dihydrate | 45.2 |
| Sodium lauryl sulfate | 1.5 |
| Flavor | 1.0 |
| Blue Color Solution | 0.05 |
| De-ionized and UV treated Water | Q.S. |

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

What is claimed is:

1. A method of reducing one or more free radical species in an oral cavity of a mammalian subject, the method comprising contacting an oral composition comprising an anti-oxidant active ingredient comprising at least one free-B-ring flavonoid and an orally acceptable carrier with an oral surface in the mammalian subject.

2. A method according to claim 1, wherein said concentration of said anti-oxidant active ingredient in said oral composition is from between about 0.001 to about 10%.

3. A method according to claim 1, wherein said concentration of said anti-oxidant active ingredient in said oral composition is from between about 0.01 to about 3%.

4. A method according to claim 1, wherein said concentration of said anti-oxidant active ingredient in said oral composition is from between about 0.1 to about 1%.

5. A method according to claim 1, wherein said anti-oxidant active ingredient is selected from the group consisting of: baicalin, baicalein, and mixtures thereof.

6. A method according to claim 1, wherein said orally acceptable carrier comprises one or more oral active ingredients selected from the group consisting of: anti-tartar agents, antibacterial agents, anti-inflammatory agents, anticaries agents, whitening agents, densensitizing agents, vitamins, compatible enzymes, chlorophyll compounds, periodontal actives, breath freshening agents, malodour control agents, salivary stimulants and combinations thereof.

7. A method according to claim 1, wherein said orally acceptable carrier comprises one or more components selected from the group consisting of: viscosity modifiers, diluents, surface active agents, pH modifying agents, abrasives, humectants, mouth feel agents, sweetening agents, flavor agents, colorants, preservatives, and combinations thereof.

8. A method according to claim 1, wherein the free radicals are produced in the oral cavity of the mammalian subject as a result of an immune response to oral pathogens.

9. A method according to claim 8, wherein said immune response is associated with a condition selected from the group consisting of gingivitis and periodontitis.

10. A method according to claim 1, wherein said contacting is repeated for a plurality of days.

11. A method of providing an anti-oxidant to an oral cavity of a mammalian subject, the method comprising contacting oral tissue in the oral cavity with an oral composition comprising an anti-oxidant active ingredient comprising a free-B-ring flavonoid and an orally acceptable carrier.

12. A method according to claim 11, wherein said concentration of said anti-oxidant active ingredient in said oral composition is from between about 0.001 to about 10%.

13. A method according to claim 11, wherein said concentration of said anti-oxidant active ingredient in said oral composition is from between about 0.1 to about 3%.

14. A method according to claim 11, wherein said anti-oxidant active ingredient is selected from the group consisting of: baicalin, baicalein, and mixtures thereof.

15. A method according to claim 11, wherein said orally acceptable carrier comprises one or more oral active ingredients selected from the group consisting of: anti-tartar agents, antibacterial agents, anti-inflammatory agents, anticaries agents, whitening agents, densensitizing agents, vitamins, compatible enzymes, chlorophyll compounds, periodontal actives, breath freshening agents, malodour control agents, salivary stimulants and combinations thereof.

16. A method according to claim 11, wherein said orally acceptable carrier comprises one or more components selected from the group consisting of: viscosity modifiers, diluents, surface active agents, pH modifying agents, abrasives, humectants, mouth feel agents, sweetening agents, flavor agents, colorants, preservatives, and combinations thereof.

17. A method according to claim 11, wherein said contacting is repeated for a plurality of days.

18. A method of preparing an anti-oxidant oral composition, the method comprising mixing an anti-oxidant ingredient comprising at least one free-B-ring flavonoid with an orally acceptable oral composition carrier.

19. A method according to claim 18, wherein said anti-oxidant ingredient comprises at least one free-B-ring flavonoid selected from the group consisting of: baicalin, baicalein, and mixtures thereof.

20. A method according to claim 18, wherein said anti-oxidant active ingredient is present in said oral composition at a concentration of from about 0.001 to about 10%.

21. A method according to claim 18, wherein said orally acceptable carrier comprises one or more oral active ingredients selected from the group consisting of: anti-tartar agents, antibacterial agents, anti-inflammatory agents, anticaries agents, whitening agents, densensitizing agents, vitamins, compatible enzymes, chlorophyll compounds, periodontal actives, breath freshening agents, malodour control agents, salivary stimulants and combinations thereof.

22. A method according to claim 18, wherein said orally acceptable carrier further comprises one or more components selected from the group consisting of: viscosity modifiers, diluents, surface active agents, pH modifying agents, abrasives, humectants, mouth feel agents, sweetening agents, flavor agents, colorants, preservatives, and combinations thereof.

23. A method according to claim 18, wherein the oral care composition is in an oral care form selected from the group consisting of a mouthrinse, a powder, a medicament, a dentifrice, a confectionary, and an animal product.
